(19) United States
(12) Patent Application Publication
(10) Pub. No.: US 2005/0014155 A1
(43) Pub. Date: Jan. 20, 2005

(54) High-density cell microarrays for parallel functional determinations

(76) Inventor: Wilson C Xu, New York, NY (US)

Correspondence Address: CLARK & ELBING LLP, 101 FEDERAL STREET, BOSTON, MA, 02110, US

(21) Appl. No.: 10/495,521

(22) PCT Filed: Nov. 15, 2002
(86) PCT No.: PCT/US02/36979

Related U.S. Application Data
(60) Provisional application No. 60/331,502, filed on Nov. 16, 2001.

(51) Int. Cl. C12Q 1/68; G01N 33/53; G01N 33/569; C12M 1/34; B05D 3/00

(52) U.S. Cl. 435/6; 435/287.2; 427/2.11

(57) ABSTRACT

Disclosed are methods for generating high-density cell microarrays. The methods generally involve forming nanocraters on a permeable membrane surface and inoculating the nanocraters with cells, proteins, or other molecules. The high-density microarrazs of the invention are useful for large-scale, high throughput phenotypic determinations of gene activities.

### FIELD OF THE INVENTION

The invention relates to high-density cell microarrays and methods for their preparation and use.

### BACKGROUND OF THE INVENTION

The availability of full genome sequences has generated great interest in studying gene functions on a genome-wide scale. Technologies are being developed that allow for the global analysis of important macromolecules that convey the information flow from DNA to RNA to proteins in cells. For example, DNA microarrays have allowed gene expression profiling for specific cellular states, and global two-hybrid analyses have provided a glimpse of intracellular signal wiring systems in yeast. Biochemical genomics will eventually enable the genome-wide analyses of protein activities, and genomic surveys of the targets of DNA binding proteins are certain to have an impact on our understanding of regulatory circuits. Systematic gene deletion projects have also begun to provide insights into gene function on a large scale.

Since the information encoded in the genome is ultimately displayed at the cellular level as cellular traits or phenotypes, global approaches for analyzing cell phenotypes would greatly facilitate our understanding of cellular functions of genes under a variety of conditions. However, there are still several challenges to studying phenotypic manifestations of gene activities on a genomic scale. In particular, large-scale phenotypic analyses require that cells be grown in parallel and in miniaturized format without cross-contamination, which can be difficult to accomplish using conventional techniques. Thus, although whole-genome sequencing projects have generated a wealth of gene sequences from a variety of organisms, developing methods for rapidly uncovering gene regulatory circuits and their functional manifestations at the cellular level remains a major challenge.

### SUMMARY OF THE INVENTION

We have developed methods for constructing high-density cell microarrays and have demonstrated that these microarrays allow for phenotypic determinations of gene activities on a large scale. Specifically, we have found that the generation of nanocraters ranging in size from about 100 pico-liters to 1.5 nano-liters on permeable membranes allows for the creation of high-density cell microarrays. Cells inoculated into the nanocraters form individual colonies that remain confined to the nanocraters and can, therefore, be arrayed very closely together (i.e. at high density) without cross-contamination.

Accordingly, the present invention features a method of making a cell microarray, which method involves generating nanocraters on a membrane surface and introducing at least one cell into the nanocraters. The membrane is incubated in a growth solution to form colonies in the nanocraters. The method thus produces a high-density microarray composed of a permeable, flexible membrane having a plurality of colonies contained within nanocraters on the membrane surface.

In various preferred embodiments of the invention, the membrane is a cellulose ester membrane; the nanocraters are generated using a robotic arrayer that simultaneous inoculates the cells into the nanocraters; and the microarray includes at least two different cell types.

A variety of cells can be arrayed using the method of the invention, including cells from plants, animals, bacteria, fungi, protozoa, and algae. In a particularly preferred embodiment the cells of the array are bacterial or mammalian. Alternatively, the method of the invention may also be used to create microarrays of various molecules, including proteins, peptides, polypeptides, nucleic acids, and lipids.

In preferred embodiments, the membrane surface is placed on a cushioning material, such as chromatography paper, during generation of the nanocraters. The cushioning material is optionally soaked with growth media to moisturize the arrayed cells and to provide nutritional or chemical requirements of the cells.

In another aspect, the invention features methods for determining phenotypic differences between cells, for determining the function of a gene, and for identifying a drug target. These methods generally involve the steps of: (a) providing a membrane surface composed of a plurality of nanocraters, wherein at least one nanocrater contains a first cell type and at least one nanocrater contains a second cell type; (b) exposing the membrane to a test substrate; (c) detecting the response of the first cell type and the second cell type to the test substrate; and (d) comparing the response of the first cell type and the second cell type. The first and second cell types may include cells of the same genus and species or may include cells that differ in one or more genes.

The test substrate may be any agent that is able to differentiate cells based on biochemical characteristics. Examples of testing substrates include, but are not limited to, carbon sources, nitrogen sources, sulfur sources, phosphorous sources, dyes, drugs, oxidizing agents, reducing agents, mutagens, amino acid analogs, sugar analogs, nucleoside analogs, base analogs, detergents, toxic metals, inorganics, antimicrobials, amino peptidase substrates, and carboxy peptidase substrates.

Typically, the microarrays of the invention include nanocraters having a diameter less than 150 μm, preferably between about 10 μm and about 125 μm. In other embodiments, the distance between the centers of adjacent nanocraters is between 400 μm and 200 μm, preferably about 375 μm. Generally, the nanocraters have a volume between about 1.5 nano-liters and about 100 pico-liters and a depth of between about 10 μm and about 125 μm.

The invention further provides cell microarrays composed of a plurality of cell colonies on a membrane surface at a density of at least 5 colony spots/mm². In various preferred embodiments, the microarrays of the invention have a density of at least 7.2 colony spots/mm², at least 10 colony spots/mm², at least 13.5 colony spots/mm², at least 100 colony spots/mm², 500 colony spots/mm², or about 1,000 colony spots/mm².

Using the methods of the invention, bacterial and yeast cell microarrays, as well as other types of microarrays, can be created that allow for phenotypic determinations of gene activities and identification of drug targets on a large scale. Such cell microarrays are particularly useful tools for studying phenotypes of gene activities on a genome-wide scale.

Other advantages and features of the present invention will be apparent from the following detailed description thereof and from the claims.

### Definitions

By “membrane” is meant a deformable, yet durable, solid support. The membrane is preferably made of a porous or permeable, flexible, water-insoluble material.

By “microarray” is meant a fixed pattern or collection of at least two different objects (e.g., cells, colonies, proteins, small molecules, etc.) that are associated with the surface of a solid support. Preferably, the array includes at least one hundred, more preferably, at least one thousand, and, most preferably, at least one hundred thousand different members.

By “nanocrater” is meant a pit, depression, or indentation in a membrane material or other deformable solid support with a volume that is on the scale of nano-liters or pico-liters. Preferably, the nanocraters have a size between about 100 pico-liters to about 1.5 nano-liters.

By “exposing” is meant allowing contact to occur between two compositions.

The term “organism” is used herein to refer to any species or type of multicellular or single-cell organism, including but not limited to, bacteria (archaebacteria, eubacteria), fungi (e.g., yeast, molds, etc.), protozoa, algae, plants, and animals, including mammals.

The terms “protein,” “polypeptide,” and “peptide” are used interchangeably herein and refer to any chain of two or more naturally occurring or modified amino acids joined by one or more peptide bonds, regardless of post-translational modification (e.g., glycosylation or phosphorylation).

By “test substrate” is meant a substance, such as a nutrient source (e.g., carbon, nitrogen, sulfur, phosphorous sources), that may be used to differentiate cells based on biochemical characteristics. For example, one bacterial organism may utilize one test substrate that is not utilized by another bacterial organism. This difference in the utilization of the test substrate may be used to differentiate between these two organisms. In certain embodiments of the invention, numerous test substrates may be used in combination.

Following exposure to a test substrate, such as a carbon or nitrogen source, or an antimicrobial agent, the response of the cells may be detected. This detection may be visual (i.e. by eye) or accomplished with the assistance of a machine. For example, growth (i.e. cell proliferation), or lack thereof, can be used as an indicator that an organism is or is not inhibited by certain anti-microbial agents. In some embodiments, colorimetric indicators (e.g. chromogenic substrates, oxidation-reduction or redox indicators, pH indicators, etc.) are used, for example, to detect the presence or absence of growth, metabolism, or other biochemical activities. Other useful test substrates include, for example, drugs, small compounds, or candidate interacting molecules (such as candidate interacting proteins, antibodies, artificial proteins, artificial nuceotides, and any other molecules.

### BRIEF DESCRIPTION OF THE DRAWINGS

FIGS. 1A-C are schematic diagrams showing the coupled fabrication of cell microarrays. (A) A robot-controlled pin with cells on the tip is programmed to strike a cellulose ester membrane to form nanocraters and simultaneously inoculate cells into them. The cellulose ester membrane is placed on top of a cushion during the arraying process. (B) Shows a view of arrayed nanocraters from above. The diameter of the nanocraters is, for example, 125 μm with a depth of 10-125 μm. The distance between centers of the adjacent nanocraters is 375 μm. (C) Cell microarray membranes are incubated on the surface of agar or liquid medium.

FIG. 2 is a photograph of nanocraters with cells at their bottoms.

FIGS. 3A-D are a series of photographs of cell microarrays of E. coli and S. cerevisiae. (A) E. coli cell microarrays (144 colonies) expressing β-galactosidase in the presence of S-gal (3,4-cyclohexenoesculectin-β-D-galactopyranoside), a chromogenic substrate for β-galactosidase. (B) E. coli cell microarrays with 48 colonies that did not express β-galactosidase. (C)S. cerevisiae microarrays grown on synthetic medium without leucine. (D) S. cerevisiae microarrays grown on synthetic medium without tryptophan.

FIG. 4 is a series of photographs of S. cerevisiae cell microarrays for assaying drug effects. 94 yeast homozygous deletion strains including an fkb1 strain and two negative controls were arrayed with 5 repeats in a total of 576 nanocraters. One set of cell microarrays was grown on YPD, the other on YPD containing 1 μg/ml of rapamycin.

### DETAILED DESCRIPTION

We have developed a unique method for generating cell microarrays that involves forming nanocraters on the surface of a solid support, such as a membrane. The nanocraters are inoculated with cells and the solid support is incubated in a growth solution to form colonies within the nanocraters. The inoculated cells proliferate to fill each nanocrater, forming one colony per nanocrater. The proliferation of cells in the nanocraters allows for the colonies to be maintained in an ordered array, making it possible to generate cell microarrays having a relatively high density. Such microarrays facilitate high-throughput screening for identification of gene functions and drug targets.

A number of solid and semi-solid materials can be used to construct the cell microarrays of the invention. Preferably, the solid support is made of a transparent material to allow for microscopic visualization of phenotypes. It should also be durable, yet flexible enough to allow cell growth under a variety of growth conditions. In addition, necessary nutrients, chemical compounds, and macromolecules should be accessible to the cells so that exogenous molecules affecting particular biological processes can be identified.

We have found that cellulose ester permeable membranes have a number of desirable properties that make these membranes a preferred material for use as a solid support for growing cells. Cellulose ester membranes are largely transparent, relatively inert, and therefore unlikely to interfere with subsequent functional assays. In addition, nutrients, small molecule compounds, and large macromolecules can freely permeate across the membranes with defined pore sizes. In fact, these membranes have routinely been used as a dialysis barrier with defined molecular weight cut-off points. Furthermore, because of their density and hydrophobicity, the membranes can float on the surface of liquid media. Moreover, liquid droplets generally do not form on the surface of the membranes after being placed on the top of agar or liquid media, which is important for preventing the flooding and subsequent cross-contamination of arrayed cells. Other materials that possess similar properties can also be used as a solid support for growing cells.

To array cells at high-density on the membranes, we developed a coupled fabrication process using a precision robot. Robot-controlled pins are first loaded with cell suspension (about 30 pico-liters) in their tips (125 μm in diameter), and programmed to strike the membrane with a pre-calibrated impact depth to form nanocraters. The size of the nanocraters typically ranges from 100 pico-liters to 1.5 nano-liters, depending on the pre-calibrated impact depth and pin size. With smaller pins, it is possible to construct smaller craters that are less than 100 pico-liters in size.

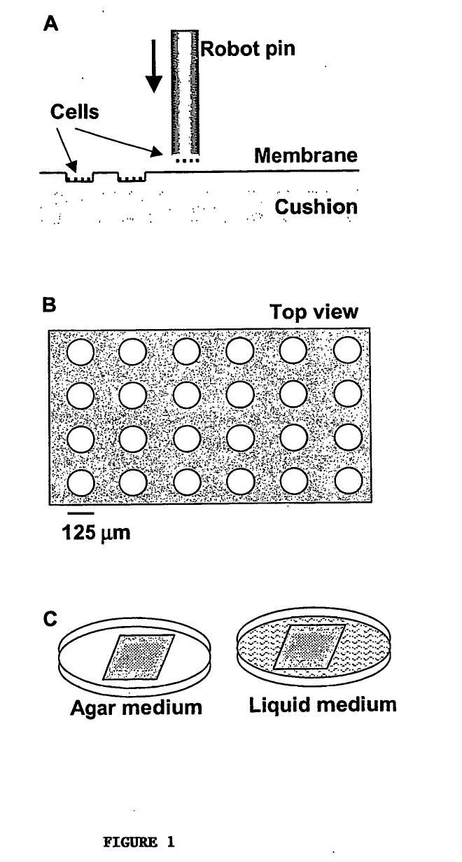

While forming the nanocraters, the robot pins simultaneously inoculate cells at the bottom of the nanocraters (see FIG. 1). Throughout the arraying process, the membranes are preferably cushioned by, for example, a piece of flat chromatography paper placed on the top of a microscope slide. The cushion prevents cellular damage from the impact and allows for the efficient formation of the nanocraters. The cushion may be soaked with growth media so that nutritional or chemical requirements of the arrayed cells are provided during the arraying process. Nanocraters generated using this process are generally able to retain their original configuration even after the membrane has been incubated on the surface of agar media for 5 months or more.

Preferably, the distance between the centers of adjacent nanocraters is less than 375 μm more preferably less than 200 μm. For the microarrays described in the Examples below, the distance between the centers of adjacent nanocraters was programmed to be 375 μm, which resulted in an array density of 7.2 colonies/mm². However, with a shorter distance, nano- or pico-craters can be arrayed at even higher densities, i.e., greater than 7.2 colony spots/mm².

The Examples provided below describe the formation of bacterial and fungal (yeast) cellular microarrays using the coupled fabrication method of the present invention. Microarrays of mammalian cells can also be made using this approach. Certain adjustments and modifications to this process should be made when arraying mammalian cells, to account for the sensitive nature of these cells. In particular, certain mammalian cells will need to be treated with proteases, such as trypsin, before arraying, so that cells are in suspension, and the cushion material should be soaked with an appropriate cell growth media In addition, the flat and solid tops of the array pins should be modified to non-flat tops (like split pens) to avoid damaging the delicate mammalian cells. After arraying, cell microarray membranes are floated on the surface of mammalian tissue culture media for phenotypic analyses.

In addition to creating cellular microarrays, the coupled fabrication approach of the invention can also be used to generate microarrays of proteins, lipids, small molecules, and other biological or synthetic molecules. The advantage of using this approach is that, unlike most methods for preparing arrays of small molecules or proteins, the molecules of the array are not chemically modified when using the methods of the present invention. Conventional methods for generating protein microarrays typically require that the proteins be immobilized to a solid support for biochemical assays. (Zhu, H. et al. Analysis of yeast protein kinases using protein chips. Nat Genet 26, 283-289. (2000); Haab, B. B., Dunham, M. J. & Brown, P. O. Protein microarrays for highly parallel detection and quantitation of specific proteins and antibodies in complex solutions. Genome Biol 2 (2001); MacBeath, G. & Schreiber, S. L. Printing proteins as microarrays for high-throughput function determination. Science 289, 1760-1763. (2000)). Similarly, certain techniques for forming microarrays of small molecules involve the covalent attachment of the molecules to a solid support (Stemson et al., J. Am. Chem. Soc. 123, 1740-1747 (2001)). With such methods, there is a possibility that biochemical properties of proteins and compounds could be altered by the immobilization or covalent attachment. The present invention eliminates this concern, because the coupled fabrication of nanocraters on permeable membranes is a physical process that requires no immobilization or chemical reactions. The methods of the invention, therefore, provide an alternative approach for making protein or other biomolecular microarrays, without significantly impacting the biochemical properties of the array members.

Stock solutions of the bio-molecules and chemical compounds can be arrayed onto the membranes using the methods and techniques described above for cells. The pore size of permeable membrane is adjusted depending on the size of the macromolecule or compound being arrayed to ensure that these molecules will be retained within the nanocraters. With pore size of the membrane smaller than that of a test compound or molecule, the nanocraters will hold the molecules preventing them from being diffused out of the membrane.

The coupled fabrication process of the invention for forming cell microarrays on permeable membranes is simple, yet accurate and robust. We have performed proof-of-principle experiments (see Examples 1-3, below) demonstrating that high-density cell microarrays allow for parallel phenotypic assays of gene activities on a large scale. In one embodiment, a real-time image acquisition system could be used to digitally record cell numbers and phenotypes (e.g., budding, cell shapes, color changes, drug resistance, etc.). The data collected at the beginning and end of the experiment could then be easily compared to streamline phenotypic determinations. This would also allow for the quantification and comparison of cell proliferation rates between different strains under a variety of conditions.

The cell microarrays of the invention can facilitate the functional studies of genome-wide gene deletion projects of microorganisms (e.g., yeast, Bacillus subtilis) or other cells that are currently under way. (Winzeler, E. A. et al. Functional characterization of the S. cerevisiae genome by gene deletion and parallel analysis. Science 285, 901-906. (1999); Ross-Macdonald, P. et al. Large-scale analysis of the yeast genome by transposon tagging and gene disruption. Nature 402, 413-418. (1999); Ogasawara, N. Systematic function analysis of Bacillus subtilis genes. Res Microbiol 151, 129-134. (2000)). For example, more than 5,000 viable yeast deletion strains can be arrayed on a permeable membrane within an area of about 6 cm². Such cell microarrays allow high-throughput cellular and physiological assays of gene activities under a variety of conditions. Therefore, cell microarrays complement other functional genomic tools such as DNA microarrays, yeast two-hybrid, and proteomic approaches. (Ideker, T. et al. Integrated genomic and proteomic analyses of a systematically perturbed metabolic network. Science 292, 929-934. (2001)).

Since cell microarrays require only a small amount of medium, one could systematically examine cellular interactions with small molecules, peptides, antibodies, polysaccharides, and other large molecules, most of which are difficult or expensive to be synthesized in large quantity. Such systematic phenotypic studies may accelerate the discovery of drug and drug targets (Hartwell, L. H., Szankasi, P., Roberts, C. J., Murray, A. W. & Friend, S. H. Integrating genetic approaches into the discovery of anticancer drugs. Science 278, 1064-1068. (1997); Mayer, T. U. et al. Small molecule inhibitor of mitotic spindle bipolarity identified in a phenotype-based screen. Science 286, 971-974. (1999)). With high-capacity diversity-oriented synthesis of small molecules, it is feasible to assay one compound (from one bead) against the cell microarrays containing the genome-wide collection of gene deletion or over-expression strains. (Tallarico, J. A. et al. An alkylsilyl-tethered, high-capacity solid support amenable to diversity-oriented synthesis for one-bead, one-stock solution chemical genetics. J Comb Chem 3, 312-318. (2001)). Cell microarrays may thus become a powerful tool in the emerging field of chemical genomics (Giaever, G. et al. Genomic profiling of drug sensitivities via induced haploinsufficiency. Nat Genet 21, 278-283. (1999)).

The present invention thus provides useful, practical, efficient and cost-effective methods for the direct and simultaneous analysis of cells and cell lines for thousands of phenotypes. The methods and microarrays of the present invention are particularly suited for analysis of phenotypic differences between various strains of organisms, including cultures that have been designated as the same genus and species, and can be used to determine the function of genes of interest. The invention can be used for phenotypic analysis and comparison of eukaryotic (e.g., fungal and mammalian), as well as prokaryotic (e.g., eubacterial and arachaebacterial) cells. For example, phenotypic differences among cells can be determined by using the coupled fabrication approach described herein to construct a cell microarray with separate nanocraters containing the different types of cells to be compared. This microarray is then exposed to a test substrate (e.g., nutrient source, antimicrobial agent, etc.) that is able to differentiate cells based on biochemical characteristics. The various responses of the cells to the test substrate are then compared to determine phenotypic differences among the cells.

The features and other details of the invention will now be more particularly described and pointed out in the following examples describing preferred techniques and experimental results. These examples are provided for the purpose of illustrating the invention and should not be construed as limiting.

### EXAMPLE 1

### Preparation of E. coli Cell Microarrays

To illustrate the coupled fabrication process, we first made cell microarrays of E. coli that express β-galactosidase. A cellulose ester membrane (Spectrum) was rinsed with and stored in deionized water at 4° C. until use. The molecular weight cut off for the membrane was 3,500 Daltons and the thickness was estimated to be 10 μm. The membrane was placed on the surface of chromatography paper (Fisher) that had been soaked in a warm 0.5% agarose solution and placed on a standard microscope slide. This cushion helped to immobilize and moisturize the membrane during the arraying process. Any bubbles or excessive agarose between the cushion and the membrane was removed by gently rubbing the membrane with a clean and smooth rod. The membrane assembly was then placed in the slide holder of a robotic arrayer (GMS417, Affymetrix).

An overnight bacterial culture was dispensed into a 96-well plate (Corning) and arrayed by the robot onto the membranes (bacterial cells can optionally be resuspended in 15% glycerol before arraying). The robotic arrayer (GMS 417, Affymetrix), equipped with 4 rings and 4 pins (125 pmi in diameter), was used to both generate nanocraters on the membrane surface and to inoculate cells into the nanocraters, employing a coupled fabrication approach. Each pin tip held about 30 pico-liters of cell suspension. Pins were programmed to strike the membrane with predetermined impact depth to form nanocraters and inoculate cells simultaneously in an approximately 50% relative humidity environment. The volume of the nanocraters was estimated to be from 100 pico-liters to 1.5 nano-liters, based on the pin size and typical depth of the nanocraters. To inoculate an adequate number of cells (typically hundreds) in each nanocrater, each spotting of cells required 2-6 strikes. The distance between the centers of adjacent arrayed nanocraters was programmed to be 375 μm, although distances as small as 200 μm were feasible with pins of 125 μm in diameter (the cell microarrays were stored on rich medium at 4° C. until use).

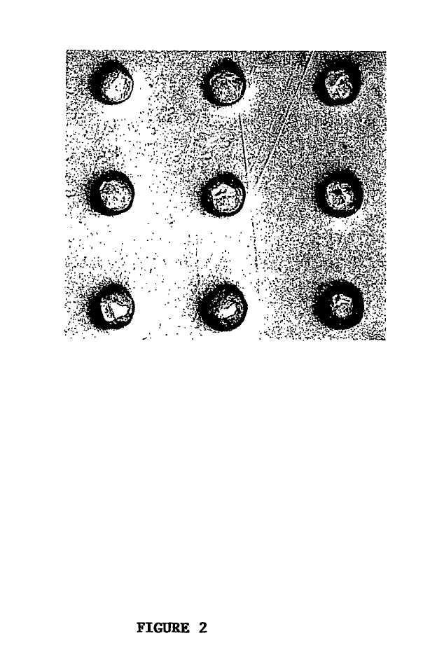

To grow cells in the nanocraters, the membranes were peeled off of the cushion and placed on the surface of rich medium containing X-gal or S-gal (Heuermann, K. & Cosgrove, J. S-Gal: an autoclavable dye for color selection of cloned DNA inserts. Biotechniques 30, 1142-1147. (2001)), chromogenic substrates of β-galactosidase. The membranes were then incubated at 37° C. overnight. Cell microarray images were captured with a microscope equipped with a digital camera. Shown in FIG. 2A are 144 colonies arrayed in an area of about 20 mm². All of the arrayed bacteria expressed β-galactosidase as indicated by the black staining. Next, we arrayed two E. coli strains, one of which expressed β-galactosidase as shown in FIG. 2B. The microarrays of LacZ⁻ cells remained white while those of LacZ⁺ cells stained black on S-gal medium.

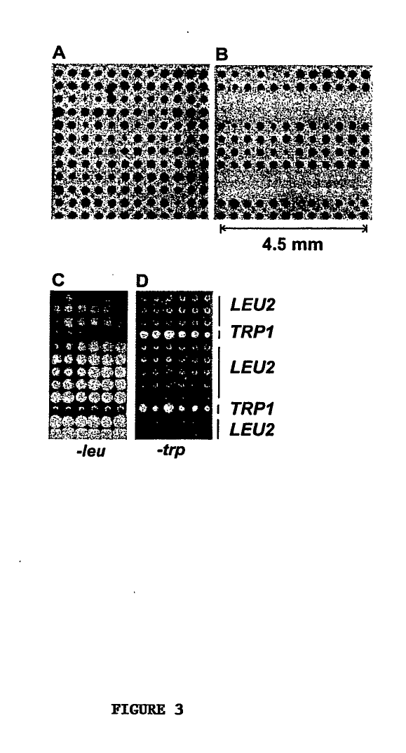

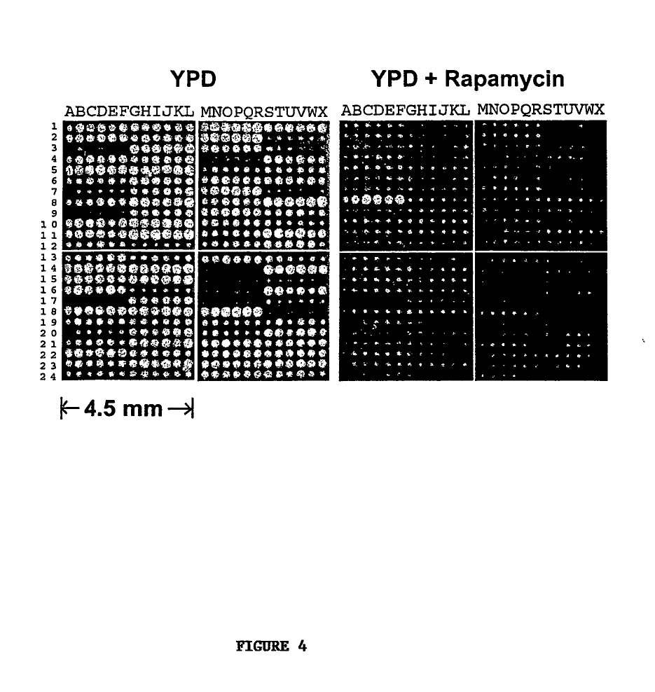

These results indicate that there was no cross-contamination between the nanocraters or even exogenous contamination during the fabrication process. Nutritional and chemical components were accessible to cells in the nanocraters on the membranes. Although cells were not homogeneously spread at the bottom of each nanocrater (FIG. 2), cells proliferated to fill each nanocrater to form one colony (FIGS. 3 and 4). As a result, the colony apices of the cell microarrays were perfectly aligned with centers of the nanocraters. These characteristics of cell proliferation in the nanocraters maintained the colonies in an ordered array (FIGS. 3 and 4), which allows for the automated storage and analyses of the cell microarray data. In contrast, if cells were arrayed onto a flat surface rather than into nanocraters, multiple colonies would form from a single arrayed spot (data not shown).

### EXAMPLE 2

### Preparation of S. cerevisiae Microarrays

Using the coupled fabrication approach described in Example 1, we next developed yeast (S. cerevisiae) cell microarrays. Two-day yeast cultures (1.2 mL) in 96-tube format (VWR) were centrifuged at 3,000 rpm for 5 min. The clear supernatant was quickly decanted without perturbing the cell pellets. About 20 μL of concentrated yeast was transferred to a 96-well plate (alternatively, the yeast cells can be resuspended in YPD+15% glycerol before arraying). Yeast cultures were dispensed into a 96-well plate and arrayed using a robotic arrayer as described above.

Shown in FIGS. 3C and 3D are yeast cell microarrays of an auxotrophic strain carrying either LEU2 or TRP1 plasmids. The cell array membranes were placed onto the surface of synthetic media lacking either leucine or tryptophan and incubated at 30° C. for 12-24 hours. Yeast cells with a LEU2 plasmid grew in the medium lacking leucine while those with a TRP1 plasmid did not. Conversely, yeast cells with a TRP1 plasmid grew on the medium lacking tryptophan but those with a LEU2 plasmid did not. These yeast results, along with the bacteria results described above, indicate that the cell microarrays allow for cellular phenotypes of genes to be conveniently and accurately assayed (cell microarray images were captured using a microscope equipped with a digital camera).

### EXAMPLE 3

### S. cerevisiae Cell Microarrays for Identifying Drug Targets

To further illustrate the utility of cell microarrays for assaying drug effects on individual gene deletions, we used a series of diploid strains carrying homozygous gene deletions of fkb1 and 93 other genes chosen at random. (Winzeler, E. A. et al. Functional characterization of the S. cerevisiae genome by gene deletion and parallel analysis. Science 285, 901-906. (1999)). FKB1 encodes FKBP12 that binds FK506 and rapamycin, two natural products used as anti-fungal and immunosuppressant drugs. The FKBP-drug complex inhibits progression through the G1 phase of the cell cycle in yeast and mammalian cells. Deletion of FKB1 has been shown to render yeast resistant to rapamycin. (Heitman, J., Movva, N. R. & Hall, M. N. Targets for cell cycle arrest by the immunosuppressant rapamycin in yeast. Science 253, 905-909. (1991); Schreiber, S. L. & Crabtree, G. R. Immunophilins, ligands, and the control of signal transduction. Harvey Lect 91, 99-114 (1995); Chan, T. F., Carvalho, J., Riles, L. & Zheng, X. F. A chemical genomics approach toward understanding the global functions of the target of rapamycin protein (TOR). Proc Natl Acad Sci USA 97, 13227-13232. (2000)). These yeast deletion strains were arrayed with 5 repeats, resulting in a total of 576 arrayed spots. The cell microarrays were incubated on the surface of YPD rich media with or without 1 μg/ml rapamycin until the fastest growing colonies were in contact with each other. As shown in FIG. 4, only the fkb1 strain could proliferate in the presence of rapamycin as predicted. There were some growth differences among cell arrays on YPD medium lacking the drug, which was in part due to the difference in growth rates of these strains. This data indicates that cell microarrays can be a powerful approach for assaying cellular functions of genes and drug targets on a large scale.

Chemicals, plasmids and strains. In connection with the above-described examples, S-gal and rapamycin were purchased from Sigma. Plasmids used were as follows: pcDNA3 (Invitrogen) and pUC18 (Stratagene), pJG4-5 and pCWX200. DH5α was used for arraying bacterial cell arrays. S. cerevisiae strains CWXY2 and EGY42 were used for yeast cell arrays for assaying auxotrophic growth. (Xu, C. W., Mendelsohn, A. R. & Brent, R. Cells that register logical relationships among proteins. Proc Natl Acad Sci USA 94, 12473-12478. (1997); Gyuris, J., Golemis, E., Chertkov, H. & Brent, R. Cdil, a human G1 and S phase protein phosphatase that associates with Cdk2. Cell 75, 791-803. (1993)). The comprehensive collection of yeast homozygous deletion strains was obtained from Research Genetics. DNA manipulations, bacterial and yeast transformation were according to standard protocols.

### Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention

All references, including patents, publications and patent applications, mentioned in this specification are herein incorporated by reference to the same extent as if each independent publication, patent or patent application was specifically and individually indicated to be incorporated by reference.

1-66. (Canceled)

67. A cell microarray comprising a plurality of cell colonies on a membrane surface, wherein said microarray has a density of at least 5 colony spots/mm².

68. The cell microarray of claim 67, wherein said microarray has a density of at least 100 colony spots/mm².

69. The cell microarray of claim 68, wherein said microarray has a density of about 1,000 colony spots/mm².

70. The cell microarray of claim 67, wherein said membrane is a permeable, flexible membrane having a plurality of nanocraters on its surface and at least one cell within said nanocraters.

71. The microarray of claim 70, wherein said membrane is a cellulose ester membrane.

72. The microarray of claim 70, wherein the distance between the centers of adjacent nanocraters is between 400 μm and 200 μm.

73. The microarray of claim 72, wherein the distance between the centers of adjacent nanocraters is about 375 μm.

74. The microarray of claim 70, wherein said nanocraters have a volume between about 1.5 nano-liters and about 100 pico-liters.

75. The microarray of claim 70, wherein said nanocraters are less than 100 pico-liters in size.

76. The microarray of claim 70, wherein said nanocraters have a diameter of 125 μm or less.

77. The microarray of claim 70, wherein said nanocraters have a diameter of about 125 μm.

78. The microarray of claim 70, wherein said nanocraters have a depth of between about 10 μm and about 125 μm.

79. The microarray of claim 67, wherein said cell is from an organism selected from the group consisting of bacteria, fungi, protozoa, and algae.

80. The microarray of claim 67, wherein said cell is mammalian.

81. The microarray of claim 67, wherein said microarray comprises at least two different cell types.

82. The microarray of claim 81, wherein said cell types comprise cells of the same genus and species.

83. The method of claim 81, wherein said cell types comprise cells that differ in one or more genes.

84. A method of identifying a drug target, said method comprising the steps of:
(a) providing a membrane surface comprising a plurality of nanocraters, wherein at least one nanocrater contains a first cell type and at least one nanocrater contains a second cell type;
(b) exposing said membrane to a test substrate;
(c) detecting the response of said first cell type and said second cell type to said test substrate; and
(d) comparing the response of said first cell type and said second cell type.

85. A method of determining the function of a gene, said method comprising the steps of:
(a) providing a membrane surface comprising a plurality of nanocraters, wherein at least one nanocrater contains a first cell type and at least one nanocrater contains a second cell type, wherein said first and second cell types differ in at least one gene;
(b) exposing said membrane to a test substrate;
(c) detecting the response of said first cell type and said second cell type to said test substrate; and
(d) comparing the response of said first cell type and said second cell type.
